(19) United States
(12) Patent Application Publication
(10) Pub. No.: US 2021/0220675 A1
(43) Pub. Date: Jul. 22, 2021

(54) RADIATION THERAPY APPARATUS, MEDICAL IMAGE PROCESSING APPARATUS, AND MEDICAL IMAGE PROCESSING METHOD

(71) Applicant: CANON MEDICAL SYSTEMS CORPORATION, Otawara-shi (JP)

(72) Inventor: Masanori KOYAMA, Nasushiobara (JP)

(73) Assignee: CANON MEDICAL SYSTEMS CORPORATION, Otawara-shi (JP)

(21) Appl. No.: 17/155,143

(22) Filed: Jan. 22, 2021

(30) Foreign Application Priority Data
Jan. 22, 2020 (JP) 2020-008033

(51) Int. Cl. A61N 5/10; G06T 5/00; A61B 5/055; A61B 5/00

(52) U.S. Cl. CPC A61N 5/1067; G06T 5/001; A61B 5/055; G06T 2207/20081; A61B 5/7264; G06T 2207/10088; A61B 5/0036

(57) ABSTRACT

A radiation therapy apparatus according to an embodiment includes processing circuitry configured: to obtain a first Magnetic Resonance (MR) image corresponding to time when radiation is being irradiated; and to perform, on the first MR image, an image processing process to reduce an impact of the radiation, so as to obtain a second MR image in which the impact of the radiation is reduced.

### CROSS-REFERENCE TO RELATED APPLICATIONS

This application is based upon and claims the benefit of priority from Japanese Patent Application No. 2020-008033, filed on Jan. 22, 2020, the entire contents of all of which are incorporated herein by reference.

### FIELD

Embodiments described herein relate generally to a radiation therapy apparatus, a medical image processing apparatus, and a medical image processing method.

### BACKGROUND

In recent years, attention has been paid to an MRI-integrated radiation therapy apparatus (which may be called “MR-Linac”) provided with a Magnetic Resonance Imaging (MRI) function and a radiation therapy (which may be called Linear Accelerator [Linac]) function. This type of radiation therapy apparatus is configured to treat a target site (e.g., a tumor) inside an examined subject by irradiating radiation such as X-rays onto the target site, while taking an MR image using the MRI function.

Further, known as techniques to assist the Linac function are Image-Guided RadioTherapy (IGRT) and synchronized irradiation. IGRT is a technique by which radiation is irradiated while checking and tracking the position of a target site, by using an image taken immediately before the irradiation or during the irradiation of the radiation. Further, the synchronized irradiation is a technique by which, for example, radiation is irradiated in synchronization with respiration or heartbeat motion, when the position of a target site moves in conjunction with the respiration or the heartbeat motion. Examples of the synchronized irradiation include pursuing irradiation where irradiation is performed by following the movement of a target site; and a wait-and-irradiate method by which irradiation is performed when a target site has moved to a position where the irradiation is possible.

### BRIEF DESCRIPTION OF THE DRAWINGS

FIG. 1 is a diagram illustrating an exemplary configuration of a radiation therapy apparatus according to an embodiment;

FIG. 2 is a diagram illustrating an exemplary configuration of a medical information processing apparatus according to the embodiment;

FIG. 3 is a chart illustrating processes in a learning mode and an operation mode performed by the radiation therapy apparatus and the medical information processing apparatus according to the embodiment;

FIGS. 4A and 4B are drawings for explaining MR images for a machine learning purpose according to the embodiment;

FIG. 5 is a flowchart illustrating a processing procedure performed by the medical information processing apparatus according to the embodiment;

FIG. 6 is a flowchart illustrating a processing procedure performed by the radiation therapy apparatus according to the embodiment;

FIG. 7 is a diagram illustrating an exemplary configuration of a radiation therapy apparatus 100 according to a third modification example of the embodiment;

FIG. 8 is a drawing for explaining processes performed by the radiation therapy apparatus 100 according to the third modification example of the embodiment; and

FIG. 9 is a diagram illustrating an exemplary configuration of a medical image processing apparatus according to another embodiment.

### DETAILED DESCRIPTION

One of the problems to be solved by the embodiments disclosed herein and in the drawings is to provide MR images with high quality in radiation therapy. It should be noted, however, that the problems to be solved by the embodiments disclosed herein and in the drawings are not limited to this problem. The problems corresponding to the advantageous effects exhibited by the configurations in the other embodiments described below may also be considered as other problems.

A radiation therapy apparatus according to an embodiment includes processing circuitry configured: to obtain a first Magnetic Resonance (MR) image corresponding to time when radiation is being irradiated; and to perform, on the first MR image, an image processing process to reduce an impact (bad effect) of the radiation, so as to obtain a second MR image in which the impact of the radiation is reduced.

Exemplary embodiments of a radiation therapy apparatus, a medical image processing apparatus, and a medical image processing method will be explained below, with reference to the accompanying drawings. Possible embodiments are not limited to the embodiments described below. Further, the description of each of the embodiments is, in principle, similarly appliable to any other embodiment. Embodiments

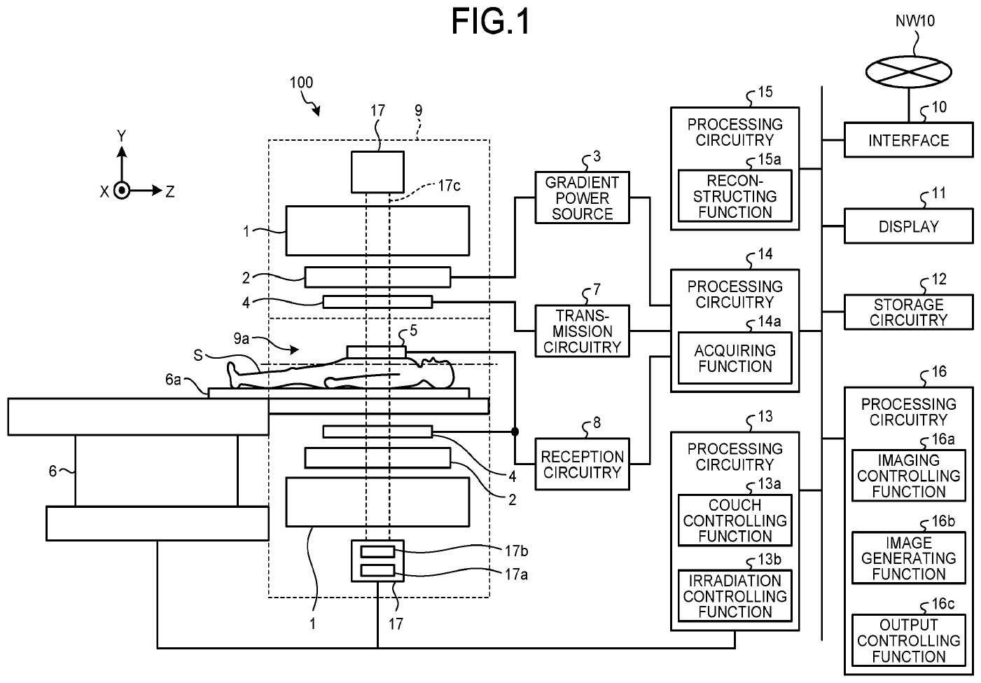

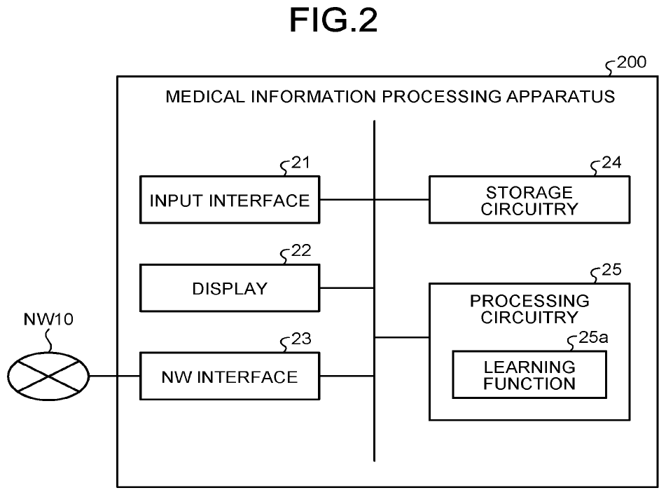

Configurations of a radiation therapy apparatus and a medical image processing apparatus according to an embodiment will be explained, with reference to FIGS. 1 and 2. FIG. 1 is a diagram illustrating an exemplary configuration of a radiation therapy apparatus 100 according to the embodiment. FIG. 2 is a diagram illustrating an exemplary configuration of a medical information processing apparatus 200 according to the embodiment.

The radiation therapy apparatus 100 illustrated in FIG. 1 is an MRI-integrated radiation therapy apparatus (which may be called “MR-Linac”) provided with a Magnetic Resonance Imaging (MRI) function and a radiation therapy (which may be called Linac) function. For example, the radiation therapy apparatus 100 is configured to assist creation of a treatment plan by taking an MR image using the MRI function, prior to the radiation therapy. Further, when the treatment plan has been created, the radiation therapy apparatus 100 is configured to perform the radiation therapy according to the treatment plan, by using the Linac function. The configuration of the radiation therapy apparatus 100 described with reference to FIG. 1 is merely an example, and possible configurations are not limited to the configuration in the drawing. For instance, a publicly-known MR-Linac configuration may arbitrarily be adopted as the configuration of the radiation therapy apparatus 100. Further, the radiation irradiated by the Linac function may be referred to as a “Linac beam” or simply a “beam”.

Further, by using the MR image, the radiation therapy apparatus 100 is capable of implementing Image-Guided RadioTherapy (IGRT) and synchronized irradiation. IGRT is a technique by which radiation is irradiated while checking and tracking the position of a target site (e.g., a tumor), by using an image taken immediately before the irradiation or during the irradiation of the radiation. Further, the synchronized irradiation is a technique by which, for example, radiation is irradiated in synchronization with respiration or heartbeat motion, when the position of a target site moves in conjunction with the respiration or the heartbeat motion. To IGRT and the synchronized irradiation, it is possible to arbitrarily apply any of publicly-known techniques.

Further, with reference to FIGS. 1 and 2, an example will be explained in which the radiation therapy apparatus 100 and the medical information processing apparatus 200 are communicably connected with each other via a network NW10; however, possible embodiments are not limited to this example. For instance, without going through the network NW10, the radiation therapy apparatus 100 and the medical information processing apparatus 200 are capable of exchanging information with each other, via a storage medium, a detachable external storage device, or the like.

The radiation therapy apparatus 100 according to the present embodiment includes a trained model and is able to provide high-quality MR images in radiation therapy, by using the trained model. Further, the medical information processing apparatus 200 according to the present embodiment is configured to construct the trained model included in the radiation therapy apparatus 100. In the present embodiment, an example will be explained in which the radiation therapy apparatus 100 includes the trained model; however, possible embodiments are not limited to this example. Another embodiment in which an apparatus different from the radiation therapy apparatus 100 includes the trained model will be explained later.

As illustrated in FIG. 1, for example, the radiation therapy apparatus 100 includes a static magnetic field magnet 1, a gradient coil 2, a gradient power source 3, a Whole Body (WB) coil 4, a reception coil device 5, a couch 6, transmission circuitry 7, reception circuitry 8, a gantry 9, an interface 10, a display 11, storage circuitry 12, processing circuitry 13, 14, 15, and 16, and a rotating frame 17. Further, the radiation therapy apparatus 100 is capable of communicating with other apparatuses connected via the network NW10. The network NW10 is an arbitrary communication network such as the Internet, a Wide Area Network (WAN), or a Local Area Network (LAN). Further, the radiation therapy apparatus 100 does not include an examined subject (hereinafter, “patient”) S (e.g., a human body) or the network NW10.

The static magnetic field magnet 1 is configured to generate a static magnetic field in an image taking space in which the patient S is placed. More specifically, the static magnetic field magnet 1 is formed to have a hollow and substantially circular cylindrical shape (which may have an oval cross-section orthogonal to the central axis thereof) and is configured to generate the static magnetic field in the image taking space formed on the inner circumferential side thereof. For example, the static magnetic field magnet 1 includes a cooling container formed to have a substantially circular cylindrical shape and a magnet such as a superconductive magnet immersed in a cooling member (e.g., liquid helium) filling the cooling container. Alternatively, for example, the static magnetic field magnet 1 may be configured to generate the static magnetic field by using a permanent magnet.

The gradient coil 2 is configured to generate gradient magnetic fields in the image taking space in which the patient S is placed. More specifically, the gradient coil 2 is formed to have a hollow and substantially circular cylindrical shape (which may have an oval cross-section orthogonal to the central axis thereof) and includes a plurality of gradient coils each having a substantially circular cylindrical shape and being stacked in the radial direction. In the present example, on the basis of an electric current supplied from the gradient power source 3, the plurality of gradient coils are configured to generate the gradient magnetic fields extending along the axial directions of the X-, Y-, and Z-axes that are orthogonal to one another, within the image taking space arranged on the inner circumferential side thereof.

More specifically, the gradient coil 2 includes: an X coil configured to generate a gradient magnetic field along the X-axis direction; a Y coil configured to generate a gradient magnetic field along the Y-axis direction; and a Z coil configured to generate a gradient magnetic field along the Z-axis direction. In this situation, the X-axis, the Y-axis, and the Z-axis structure an apparatus coordinate system unique to the radiation therapy apparatus 100. For example, the X-axis is set in a horizontal direction orthogonal to the central axis of the gradient coil 2. The Y-axis is set in a vertical direction orthogonal to the central axis of the gradient coil 2. The Z-axis is set along the central axis of the gradient coil 2.

By individually supplying an electric current to each of the X, Y, and Z coils included in the gradient coil 2, the gradient power source 3 is configured to cause the gradient magnetic fields along the axial directions of the X-, Y-, and Z-axes to be generated within the image taking space. More specifically, by supplying the electric current to each of the X, Y, and Z coils as appropriate, the gradient power source 3 is configured to cause the gradient magnetic fields to be generated along a readout direction, a phase encode direction, and a slice direction, respectively, that are orthogonal to one another. In this situation, the axis along the readout direction, the axis along the phase encode direction, and the axis along the slice direction structure a logical coordinate system used for defining a slice region or a volume region subject to the imaging process.

In the following sections, an example will be explained in which the axis extending along the readout direction, the axis extending along the phase encode direction, and the axis extending along the slice direction which structure the logical coordinate system correspond to the X-axis, the Y-axis, and the Z-axis, respectively, which structure the apparatus coordinate system. However, possible correspondence relationships between the logical coordinate system and the apparatus coordinate system are not limited to the relationship in this example, and it is possible to change the relationship arbitrarily.

Further, as each being superimposed on the static magnetic field generated by the static magnetic field magnet 1, the gradient magnetic fields along the readout direction, the phase encode direction, and the slice direction append spatial position information to a Magnetic Resonance (MR) signal emitted from the patient S. More specifically, the gradient magnetic field Gro in the readout direction appends position information along the readout direction to the MR signal, by changing the frequency of the MR signal in accordance with the position in the readout direction. Further, the gradient magnetic field GPe in the phase encode direction appends position information along the phase encode direction to the MR signal, by changing the phase of the MR signal along the phase encode direction. Further, the gradient magnetic field Gss in the slice direction appends position information along the slice direction to the MR signal. For example, the gradient magnetic field Gss in the slice direction is used for determining the orientations, the thicknesses, and the quantity of slice regions when imaged regions are the slice regions and is used for changing the phase of the MR signal in accordance with the position in the slice direction when an imaged region is a volume region.

The WB coil 4 is arranged on the inside of the gradient coil 2 and is a Radio Frequency (RF) coil having a function of a transmission coil configured to apply a Radio Frequency (RF) magnetic field to the image taking space in which the patient S is placed and a function of a reception coil configured to receive the MR signal emitted from the patient S due to influence of the RF magnetic field. More specifically, the WB coil 4 is formed to have a hollow and substantially circular cylindrical shape (which may have an oval cross-section orthogonal to the central axis thereof) and is configured to apply the RF magnetic field to the image taking space arranged inside the circular cylinder, on the basis of a radio frequency pulse signal supplied from the transmission circuitry 7. Further, the WB coil 4 is configured to receive the MR signal emitted from the patient S due to the influence of the RF magnetic field and to output the received MR signal to the reception circuitry 8.

The reception coil device 5 is an RF coil configured to receive the MR signal emitted from the patient S. For example, the reception coil device 5 is prepared for each site of the patient S and, at the time of imaging the patient S, is arranged in the vicinity of the site to be imaged. Further, the reception coil device 5 is configured to receive the MR signal emitted from the patient S due to the influence of the RF magnetic field applied by the WB coil 4 and to output the received MR signal to the reception circuitry 8. Further, the reception coil device 5 may also have the function of a transmission coil configured to apply the RF magnetic field to the patient S. In that situation, the reception coil device 5 is connected to the transmission circuitry 7 and is configured to apply the RF magnetic field to the patient S on the basis of the RF pulse signal supplied from the transmission circuitry 7.

The couch 6 includes a couchtop 6a on which the patient S is placed. When an imaging process is to be performed on the patient S, the couchtop 6a on which the patient S is placed is moved into the image taking space. For example, the couch 6 is installed so that the longitudinal direction of the couchtop 6a extends parallel to the central axis of the static magnetic field magnet 1.

The transmission circuitry 7 is configured to output the RF pulse signal corresponding to a resonance frequency (a Larmor frequency) unique to targeted atomic nuclei placed in the static magnetic field, to the WB coil 4. More specifically, the transmission circuitry 7 includes a pulse generator, an RF generator, a modulator, and an amplifier. The pulse generator is configured to generate a waveform of the RF pulse signal. The RF generator is configured to generate an RF signal having the resonance frequency. The modulator is configured to generate the RF pulse signal by modulating the amplitude of the RF signal generated by the RF generator, with the waveform generated by the pulse generator. The amplifier is configured to amplify the RF pulse signal generated by the modulator and to output the amplified signal to the WB coil 4.

The reception circuitry 8 is configured to generate MR signal data on the basis of the MR signal received by either the WB coil 4 or the reception coil device 5. For example, the reception circuitry 8 is configured to generate the MR signal data by digitally converting the MR signal output from either the WB coil 4 or the reception coil device 5. Further, the reception circuitry 8 is configured to output the generated MR signal data to the processing circuitry 14.

The gantry 9 has a hollow bore 9a formed to have a substantially circular cylindrical shape (which may have an oval cross-section orthogonal to the central axis thereof) and is configured to support the static magnetic field magnet 1, the gradient coil 2, and the WB coil 4. More specifically, the gantry 9 is configured to support the static magnetic field magnet 1, the gradient coil 2, and the WB coil 4, while the gradient coil 2 is arranged on the inner circumferential side of the static magnetic field magnet 1, the WB coil 4 is arranged on the inner circumferential side of the gradient coil 2, and the bore 9a is arranged on the inner circumferential side of the WB coil 4. In this situation, the space inside the bore 9a of the gantry 9 is the image taking space in which the patient S is placed when the imaging process is performed on the patient S.

The example is explained in which the radiation therapy apparatus 100 has a so-called tunnel-like structure in which the static magnetic field magnet 1, the gradient coil 2, and the WB coil 4 are each formed to have the substantially cylindrical shape; however, possible embodiments are not limited to this example. For instance, the radiation therapy apparatus 100 may have a so-called open structure in which a pair of static magnetic field magnets, a pair of gradient coil units, and a pair of RF coils are arranged so as to oppose each other, while the image taking space in which the patient S is placed is interposed therebetween. In that situation, the spaced interposed between the pair of static magnetic field magnets, the pair of gradient coil units, and the pair of RF coils corresponds to the bore in the tunnel-like structure.

The interface 10 is configured to receive operations to input various types of instructions and various types of information from the operator. More specifically, the interface 10 is connected to the processing circuitry 16 and is configured to convert the input operations received from the operator into electrical signals and to output the electrical signals to the processing circuitry 16. For example, the interface 10 includes a trackball, a switch button, a mouse, a keyboard, a touchpad on which an input operation can be performed by touching the operation surface thereof, a touch screen in which a display screen and a touchpad are integrally formed, contactless input circuitry using an optical sensor, audio input circuitry, and/or the like that are used for setting image taking conditions, a Region of Interest (ROI), and the like. In the present disclosure, the interface 10 does not necessarily have to include one or more physical operational component parts such as a mouse, a keyboard, and/or the like. Examples of the interface 10 include, for instance, electrical signal processing circuitry configured to receive an electrical signal corresponding to an input operation from an external input device provided separately from the apparatus and to output the electrical signal to control circuitry.

Further, the interface 10 is configured to control communication between the radiation therapy apparatus 100 and the medical information processing apparatus 200. More specifically, the interface 10 is configured to receive various types of information from the medical information processing apparatus 200 and to output the received information to the processing circuitry 16. For example, the interface 10 includes a network card, a network adaptor, a Network Interface Controller (NIC), or the like.

The display 11 is configured to display various types of information and various types of images. More specifically, the display 11 is connected to the processing circuitry 16 and is configured to convert various types of information and various types of images sent thereto from the processing circuitry 16 into display-purpose electrical signals and to output the electrical signals. For example, the display 11 is realized by using a liquid crystal monitor, a Cathode Ray Tube (CRT) monitor, a touch panel, or the like.

The storage circuitry 12 is configured to store various types of data therein. More specifically, the storage circuitry 12 is configured to store therein the MR signal data and MR images. For example, the storage circuitry 12 is realized by using a semiconductor memory element such as a Random Access memory (RAM) or a flash memory, or a hard disk, an optical disk, or the like.

The processing circuitry 13 includes a couch controlling function 13a and an irradiation controlling function 13b. The couch controlling function 13a is configured to control operations of the couch 6, by outputting control-purpose electrical signals to the couch 6. For example, via the interface 10, the couch controlling function 13a receives, from the operator, an instruction to move the couchtop 6a in a longitudinal direction, an up-and-down direction, or a left-and-right direction and brings a moving mechanism of the couchtop 6a included in the couch 6 into operations, so as to move the couchtop 6a according to the received instruction. The irradiation controlling function 13b will be explained later.

The processing circuitry 14 includes an acquiring function 14a. The acquiring function 14a is configured to acquire the MR signal data of the patient S, by executing any of various types of pulse sequences. More specifically, the acquiring function 14a is configured to execute a pulse sequence by driving the gradient power source 3, the transmission circuitry 7, and the reception circuitry 8 according to sequence execution data output from the processing circuitry 16. In this situation, the sequence execution data is data representing the pulse sequence and is information that defines: the timing with which the electric current is to be supplied by the gradient power source 3 to the gradient coil 2 and the intensity of the electric current to be supplied; the intensity of the RF pulse signal to be supplied by the transmission circuitry 7 to the WB coil 4 and the timing with which the RF pulse signal is to be supplied; the detection timing with which the MR signals are to be detected by the reception circuitry 8, and the like. Further, as a result of the pulse sequence being executed, the acquiring function 14a is configured to receive the MR signal data from the reception circuitry 8 and to store the received MR signal data into the storage circuitry 12. In this situation, a set of MR signal data received by the acquiring function 14a is stored in the storage circuitry 12 as data structuring a k-space, by being arranged two-dimensionally or three-dimensionally in accordance with the position information appended by the readout gradient magnetic field, the phase encode gradient magnetic field, and the slice gradient magnetic field described above.

The processing circuitry 15 includes a reconstructing function 15a. The reconstructing function 15a is configured to generate an MR image on the basis of the MR signal data stored in the storage circuitry 12. More specifically, the reconstructing function 15a is configured to generate the MR image by reading the MR signal data stored in the storage circuitry 12 by the acquiring function 14a and performing a post-processing process, i.e., a reconstructing process such as a Fourier transform, on the read MR signal data. Further, the reconstructing function 15a is configured to store the generated MR image into the storage circuitry 12.

The processing circuitry 16 includes an imaging controlling function 16a, an image generating function 16b, and an output controlling function 16c. The imaging controlling function 16a is configured to control the entirety of MRI processes performed by the radiation therapy apparatus 100, by controlling constituent elements relevant to MRI. More specifically, the imaging controlling function 16a causes the display 11 to display a Graphical User Interface (GUI) used for receiving operations to input various types of instructions and various types of information from the operator. Further, in accordance with the input operations received via the interface 10, the imaging controlling function 16a is configured to control the constituent elements relevant to MRI. For example, the imaging controlling function 16a is configured to receive an input of image taking conditions from the operator via the interface 10. Further, the imaging controlling function 16a is configured to generate sequence execution data on the basis of the received image taking conditions and to execute any of various types of pulse sequences by transmitting the sequence execution data to the processing circuitry 14. Further, for example, in response a request from the operator, the imaging controlling function 16a is configured to read any of the MR images from the storage circuitry 12 and to output the read MR image to the display 11. The imaging controlling function 16a is an example of an obtaining unit configured to obtain an MR image. The image generating function 16b and the output controlling function 16c will be explained later.

The processing circuitry 13, 14, 15, and 16 explained above are realized by using one or more processors, for example. In that situation, the processing functions of the processing circuitry are, for example, stored in the storage circuitry 12 in the form of computer-executable programs. Each of the pieces of processing circuitry is configured to realize the function corresponding to a different one of the programs, by reading and executing the program from the storage circuitry 12. In this situation, the pieces of processing circuitry may be configured by using two or more processors, so that the processing functions are realized as a result of the processors executing the programs. Alternatively, the processing functions of the pieces of processing circuitry may be realized as being distributed among or integrated into one or more pieces of processing circuitry as appropriate. Further, although the example was explained above in which the single piece of storage circuitry (i.e., the storage circuitry 12) stores therein the programs corresponding to the processing functions, another arrangement is also acceptable in which a plurality of pieces of storage circuitry are arranged in a distributed manner, so that the processing circuitry reads a corresponding program from each of the individual pieces of storage circuitry.

The rotating frame 17 is an annular frame arranged so as to enclose the static magnetic field magnet 1 and is configured to support a radiation generator 17a and a radiation limiter 17b. For example, the radiation generator 17a includes an electron gun and an accelerator tube and is configured to irradiate treatment-purpose radiation. The accelerator tube is configured to generate the treatment-purpose radiation by accelerating thermo electrons from the electron gun so as to collide with a tungsten target. The radiation limiter 17b includes a plurality of limiting blades (which may be referred to as “multi-leaf collimator”) configured to limit the irradiation range of the treatment-purpose radiation and a moving mechanism configured to move the plurality of limiting blades.

Further, by rotating on a circular trajectory centered on the patient S under the control of the irradiation controlling function 13b, the rotating frame 17 is configured to cause the radiation generator 17a and the radiation limiter 17b to rotate and move while being centered on the patient S. As a result, the radiation generated by the radiation generator 17a on the circular trajectory is irradiated onto the patient S via an irradiation path 17c. In this situation, the gantry 9 includes a driving device and the like that causes the rotating frame 17 to rotate.

To allow the radiation to reach the patient S, radiation windows are provided in the constituent elements positioned on the irradiation path 17c. The radiation windows are regions designed so that the attenuation of the radiation at the constituent elements are smallest possible and are uniform. It is possible to arbitrarily apply any of publicly-known techniques to the configuration of the radiation windows.

In this situation, by employing the irradiation controlling function 13b, the radiation therapy apparatus 100 is configured to perform radiation therapy on a target site of the patient S. The irradiation controlling function 13b is configured to control the entirety of the radiation irradiating processes performed by the radiation therapy apparatus 100, by controlling constituent elements relevant to the radiation therapy. More specifically, the irradiation controlling function 13b is configured to cause the display 11 to display a Graphical User Interface (GUI) for receiving operations to input various types of instructions and various types of information from the operator. Further, the irradiation controlling function 13b is configured to control the constituent elements relevant to the radiation therapy, in accordance with the input operations received via the interface 10.

For example, in response to a request from the operator, the irradiation controlling function 13b is configured to read any of the MR images from the storage circuitry 12 and to output the read MR image to the display 11. By referring to the MR image displayed on the display 11, the operator designates, via the interface 10, the contours of the target site and organs that are highly sensitive to radiation and need to be prevented from being irradiated with the radiation. On the basis of the information designated by the operator, the irradiation controlling function 13b is configured to analyze the three-dimensional shape and the position of the target site, as well as positional relationships thereof with the designated organs. After that, on the basis of a result of the analysis, the irradiation controlling function 13b is configured to create a treatment plan for continuing the radiation therapy on the target site of the patient S for a long period of time (e.g., a number of weeks to a number of months). For example, the treatment plan includes information about the radiation quality, the incident direction, the irradiation field, the radiation dose, the number of times of irradiation, and the like of the radiation to be used in the radiation therapy. In this situation, the treatment plan does not necessarily have to be created by the irradiation controlling function 13b. For instance, the irradiation controlling function 13b may receive and utilize a treatment plan created by a medical information processing apparatus or other medical image diagnosis apparatuses having the function of creating treatment plans. Further, to create the treatment plan, it is also acceptable to use, besides the MR images, a medical image taken by another medical image diagnosis apparatus such as a Computed Tomography (CT) image or an ultrasound image, or a combination of any of these types of medical images.

Further, according to the treatment plan, the irradiation controlling function 13b is configured to determine irradiation conditions of the radiation to be irradiated in each session of the radiation therapy. After that, on the basis of the determined irradiation conditions, the irradiation controlling function 13b is configured to irradiate the radiation on the target site of the patient S. For example, the irradiation controlling function 13b is configured to generate radiation in each of different positions on the circular trajectory, by controlling the application voltage, the application time period, and the like of a high-voltage generator of the radiation generator 17a, while rotating the rotating frame 17. Further, the irradiation controlling function 13b is configured to form a radiation irradiation region having a shape corresponding to the shape of the target site of the patient S, by controlling the positional arrangements of the plurality of limiting blades of the radiation limiter 17b in each of different positions on the circular trajectory. With these arrangements, the radiation therapy apparatus 100 is able to effectively irradiate the radiation onto the target site, while keeping as little as possible the impacts of the radiation on the normal sites (the sites other than the target site) of the patient S.

Next, a configuration of the medical information processing apparatus 200 illustrated in FIG. 2 will be explained. As illustrated in FIG. 2, the medical information processing apparatus 200 includes an input interface 21, a display 22, a network (NW) interface 23, storage circuitry 24, and processing circuitry 25.

The input interface 21 is configured to receive operations to input various types of instructions and information from the operator. More specifically, the input interface 21 is configured to convert the input operations received from the operator into electrical signals and to output the electrical signals to the processing circuitry 25. For example, the input interface 21 is realized by using a trackball, a switch button, a mouse, a keyboard, a touchpad on which an input operation can be performed by touching the operation surface thereof, a touch screen in which a display screen and a touchpad are integrally formed, contactless input circuitry using an optical sensor, audio input circuitry, and/or the like. In this situation, the input interface 21 does not necessarily have to include one or more physical operational component parts such as a mouse, a keyboard, and/or the like. Examples of the input interface 21 include, for instance, electrical signal processing circuitry configured to receive an electrical signal corresponding to an input operation from an external input device provided separately from the apparatus and to output the electrical signal to control circuitry.

The display 22 is configured to display various types of information and images. More specifically, the display 22 is configured to convert data of information and images sent thereto from the processing circuitry 25 into display-purpose electrical signals and to output the electrical signals. For example, the display 22 is realized by using a liquid crystal monitor, a Cathode Ray Tube (CRT) monitor, a touch panel, or the like. Examples of output devices provided for the medical information processing apparatus 200 are not limited to the display 22, and a speaker may be provided, for instance. The speaker may, for example, output a prescribed sound such as a beep sound, to notify the operator of a processing status of the medical information processing apparatus 200.

The NW interface 23 is connected to the processing circuitry 25 and is configured to control communication between the medical information processing apparatus 200 and external apparatuses. More specifically, via the network NW10, the NW interface 23 is configured to receive various types of information from the external apparatuses and to output the received information to the processing circuitry 25. For example, the NW interface 23 is realized by using a network card, a network adaptor, a NIC, or the like.

The storage circuitry 24 is connected to the processing circuitry 25 and is configured to store therein various types of data. For example, the storage circuitry 24 is realized by using a semiconductor memory element such as a RAM or a flash memory, or a hard disk, an optical disk, or the like.

In accordance with the input operations received from the operator via the input interface 21, the processing circuitry 25 is configured to control operations of the medical information processing apparatus 200. For example, the processing circuitry 25 is realized by using a processor.

Further, the processing circuitry 25 is configured to execute a learning function 25a. The learning function 25a is an example of a learning unit. Details of the processes performed by the learning function 25a executed by the processing circuitry 25 will be explained later.

Because it is known that MRI and Linac can have impacts on each other, MR-Linac is provided with various contrivance for reducing the impacts which the two functions may have on each other. However, conventional techniques exhibit a tendency where MR images taken while a Linac beam is being irradiated (which may be referred to as “MR images corresponding to Linac beam-ON time”) can have an image distortion or image noise more easily than MR images taken while no Linac beam is being irradiated (which may be referred to as “MR images corresponding to Linac beam-OFF time”). Further, when IGRT or synchronized irradiation is performed by using an MR image having an image distortion or image noise, there is a possibility that the precision levels of the IGRT or the synchronized irradiation may be degraded.

To cope with the circumstances described above, the radiation therapy apparatus 100 according to the present embodiment has the processing functions described below for the purpose of providing high-quality MR images in the radiation therapy. In other words, in the radiation therapy apparatus 100, the imaging controlling function 16a is configured to obtain a first Magnetic Resonance (MR) image corresponding to the Linac beam-ON time of a first patient. The image generating function 16b is configured to perform, on the first MR image, an image processing process to reduce the impacts of the Linac beam, so as to obtain a second MR image in which the impacts of the Linac beam are reduced. The second MR image is an image obtained by reducing image distortions and image noise in the first MR image. In other words, the image generating function 16b is configured to generate the second MR image corresponding to the Linac beam-OFF time, from the first MR image corresponding to the Linac beam-ON time. For example, with respect to a plurality of second patients, the image generating function 16b is configured to use a trained model that has been trained in advance by using MR images corresponding to the Linac beam-ON time and MR images corresponding to the Linac beam-OFF time (a trained model trained by using a data set in which the MR images corresponding to the Linac beam-ON time serve as training-purpose data [inputs] and the MR images corresponding to the Linac beam-OFF time serve as correct answer data [outputs]). The image generating function 16b is configured to generate the second MR image corresponding to the Linac beam-OFF time (the MR image obtained by reducing image distortions and image noise in the first MR image), by inputting the first MR image to the trained model. The output controlling function 16c is configured to output the second MR image. In the explanations below, the “MR image corresponding to the Linac beam-ON time” may be referred to as an “MR image corresponding to beam-ON time”. Also, the “MR image corresponding to the Linac beam-OFF time” may be referred to as an “MR image corresponding to beam-OFF time”.

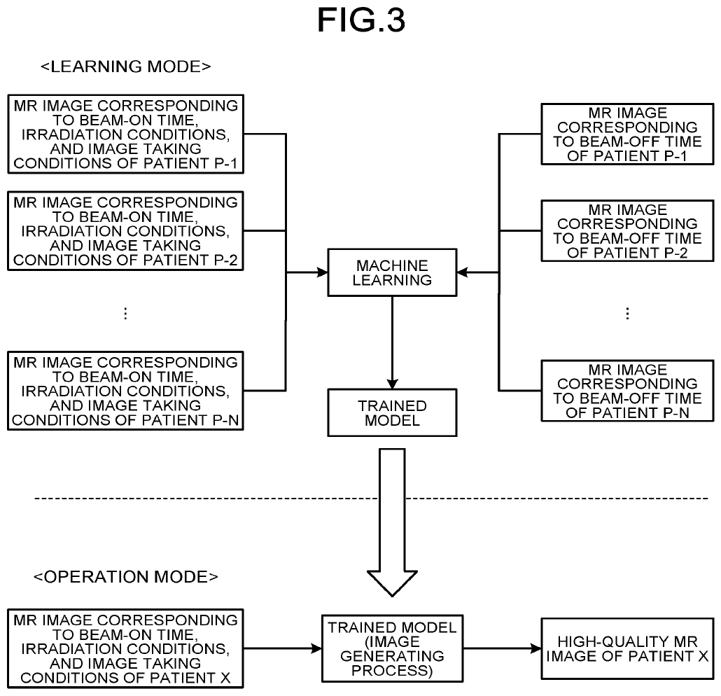

Processes performed in a learning mode and an operation mode by the radiation therapy apparatus 100 and the medical information processing apparatus 200 according to the embodiment will be explained, with reference to FIG. 3. FIG. 3 is a chart illustrating the processes in the learning mode and the operation mode performed by the radiation therapy apparatus 100 and the medical information processing apparatus 200 according to the embodiment.

As illustrated in the top section of FIG. 3, in the learning mode, the medical information processing apparatus 200 performs a machine learning process by using, for example, “MR images corresponding to the beam-ON time”, “irradiation conditions”, “image taking conditions”, and “MR images corresponding to the beam-OFF time” of patients P-1 to P-N. Details of the “MR images corresponding to the beam-ON time”, the “irradiation conditions”, the “image taking conditions”, and the “MR images corresponding to the beam-OFF time” will be explained later.

In other words, through the machine learning process that uses the “MR images corresponding to the beam-OFF time” as training data (the correct answer data), the medical information processing apparatus 200 constructs the trained model configured to output a “high-quality MR image” similar to an MR image corresponding to the beam-OFF time, in response to an input of an “MR image corresponding to the beam-ON time”, “irradiation conditions”, and “image taking conditions” of any given patient. The trained model is transferred to the radiation therapy apparatus 100 and is stored into the storage circuitry 12.

After that, as illustrated in the bottom section of FIG. 3, in the operation mode, the radiation therapy apparatus 100 causes the trained model to output a “high-quality MR image” of a patient X who is undergoing radiation therapy, by inputting an “MR image corresponding to the beam-ON time”, “irradiation conditions”, and “image taking conditions” of the patient X to the constructed trained model.

The description referencing FIG. 3 is merely an example, and possible embodiments are not limited to the example in the drawing. For instance, the “irradiation conditions” and the “image taking conditions” do not necessarily need to be input as the input data of the machine learning process. For example, the medical information processing apparatus 200 is able to perform the machine learning process, as long as it is possible to input at least “MR images corresponding to the beam-ON time” and “MR images corresponding to the beam-OFF time”, as the input data of the machine learning process. It should be noted that, when “irradiation conditions” are input as the input data in the learning mode, it is desirable to have “irradiation conditions” input in the operation mode, too. Similarly, when “image taking conditions” are input as the input data in the learning mode, it is desirable to have “image taking conditions” input in the operation mode, too.

Next, processing functions of the radiation therapy apparatus 100 and the medical information processing apparatus 200 according to the embodiment will be explained. In the following sections, at first, the medical information processing apparatus 200 configured to generate the trained model will be explained. After that, the radiation therapy apparatus 100 configured to generate the high-quality MR image by using the trained model will be explained.

In the medical information processing apparatus 200, the storage circuitry 24 has stored therein, with respect to the plurality of patients P-1 to P-N, the “MR images corresponding to the beam-ON time”, the “irradiation conditions”, the “image taking conditions”, and the “MR images corresponding to the beam-OFF time”.

Among these, the “MR images corresponding to the beam-ON time” are each an MR image taken of a different one of the patients P-1 to P-N while the Linac beam is being irradiated. The patients P-1 to P-N are examples of the second patients.

The “irradiation conditions” are irradiation conditions of the Linac beam used at the time of taking the MR images corresponding to the beam-ON time. The irradiation conditions include, for example, a beam output, a beam angle, a limiter opening degree, and an irradiation time period. Among these, the beam output indicates the output intensity of the Linac beam. The beam angle indicates the irradiation angle (the irradiation direction) of the Linac beam. The limiter opening degree indicates an extent to which the irradiation range defined by the plurality of limiting blades is open. Further, the irradiation time period indicates the time period (the duration) during which the Linac beam is irradiated. The irradiation conditions corresponding to the beam-ON time for the patients P-1 to P-N are examples of a second irradiation condition.

Further, the “image taking conditions” are image taking conditions of the MR images corresponding to the beam-ON time. The image taking conditions include, for example, a magnetic field intensity, a Field Of View (FOV), a slick thickness, an image taking time period, and a sequence. Among these, the magnetic field intensity indicates the magnetic field intensity of the static magnetic field magnet 1. The FOV indicates the position and the size of the image taking space for the MRI. The slice thickness indicates the slick thickness of the MR image. The image taking time period indicates the time period (the duration) required to take the MR image. The sequence indicates the type of the pulse sequence. The image taking conditions of the MR images corresponding to the beam-ON time for the patients P-1 to P-N are examples of a second image taking condition.

The image taking conditions of the MR images corresponding to the beam-OFF time are basically the same as the image taking conditions of the MR images corresponding to the beam-ON time; however, when the image taking conditions of the MR images corresponding to the beam-OFF time are different from the image taking conditions of the MR images corresponding to the beam-ON time, it is desirable to store the image taking conditions of the MR images corresponding to the beam-OFF time in the storage circuitry 24 so as to be used as training-purpose data.

Further, the “MR images corresponding to the beam-OFF time” are each an MR image taken of a different one of the patients P-1 to P-N, while no Linac beam is being irradiated.

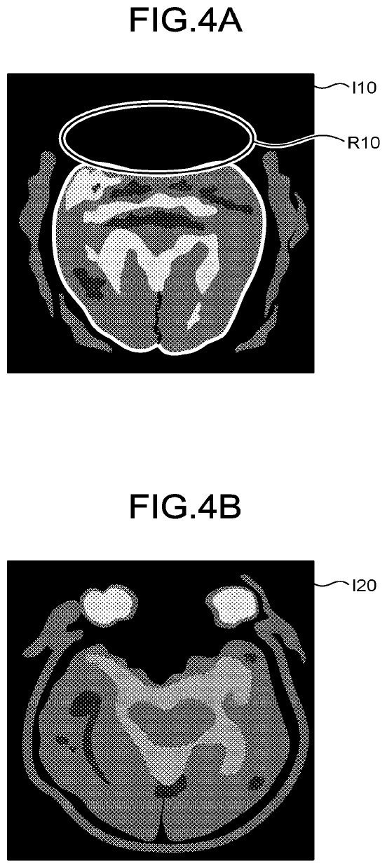

Next, the MR images for the machine learning purpose according to the embodiment will be explained, with reference to FIGS. 4A and 4B. FIGS. 4A and 4B are drawings for explaining the MR images for the machine learning purpose according to the embodiment. FIG. 4A illustrates an MR image I10 corresponding to the beam-ON time. FIG. 4B illustrates an MR image I20 corresponding to the beam-OFF time.

As illustrated in FIG. 4A, the MR image I10 corresponding to the beam-ON time has an image distortion in a region R10, due to impacts of the Linac beam. In contrast, as illustrated in FIG. 4B, the MR image I20 corresponding to the beam-OFF time has no image distortions and has higher image quality than the MR image I10 corresponding to the beam-ON time.

The description referencing FIGS. 4A and 4B is merely an example, and possible embodiments are not limited to this example. For instance, although FIG. 4A illustrates the example having the image distortion, possible embodiments are not limited to this example. For instance, an MR image corresponding to the beam-ON time may have image noise (artifacts) due to impacts of the Linac beam.

In the medical information processing apparatus 200, the learning function 25a is configured to construct the trained model by performing the machine learning process while using the MR images corresponding to the Linac beam-ON time and the MR images corresponding to the Linac beam-OFF time with respect to the plurality of patients P-1 to P-N. The constructed trained model is stored into the storage circuitry 24.

For example, the learning function 25a is configured to read, from the storage circuitry 24, the “MR images corresponding to the beam-ON time”, the “irradiation conditions”, the “image taking conditions”, and the “MR images corresponding to the beam-OFF time” of the plurality of patients P-1 to P-N. After that, on the basis of the differences (deviations) between the MR images corresponding to the beam-ON time and the MR images corresponding to the beam-OFF time, the learning function 25a is configured to quantitatively evaluate and learn impacts of the Linac beam imposed on the MR images, such as image distortions and image noise. It is possible to realize the machine learning process performed by the learning function 25a, by using a publicly-known machine learning engine, for example. Examples of applicable publicly-known machine learning engines include deep learning (a neural network) and a Support Vector Machine (SVM).

As explained above, the learning function 25a is configured to construct the trained model by performing the machine learning process on the basis of the “MR images corresponding to the beam-ON time”, the “irradiation conditions”, the “image taking conditions”, and the “MR images corresponding to the beam-OFF time” of the plurality of patients P-1 to PN. The constructed trained model is transferred to the radiation therapy apparatus 100 and stored into the storage circuitry 12.

Returning to the description of FIG. 1, in the radiation therapy apparatus 100, the imaging controlling function 16a is configured to take MR images of a region including the target site of the patient X undergoing the radiation therapy. In this situation, the MR images taken by the imaging controlling function 16a may include an MR image corresponding to the Linac beam-ON time and an MR image corresponding to the Linac beam-OFF time. In other words, the imaging controlling function 16a is an example of an obtaining unit configured to obtain the MR image corresponding to the Linac beam-ON time of the patient X. Further, the MR image corresponding to the Linac beam-ON time of the patient X is an example of the first MR image. In other words, the imaging controlling function 16a is an example of a first obtaining unit configured to obtain the first MR image corresponding to the time when radiation is being irradiated.

When the MR image has been taken during the Linac beam-ON time, the imaging controlling function 16a outputs, to the image generating function 16b, the MR image corresponding to the Linac beam-ON time, the irradiation conditions of the Linac beam irradiated at the time of taking the MR image, and the image taking conditions of the MR image. In this situation, the irradiation conditions of the Linac beam irradiated at the time of taking the MR image corresponding to the Linac beam-ON time are examples of a first irradiation condition. Further, the image taking conditions of the MR image corresponding to the Linac beam-ON time are examples of a first image taking condition.

The image generating function 16b is configured to generate the “high quality MR image” of the patient X, by inputting the “MR image corresponding to the beam-ON time”, the “irradiation conditions”, and the “image taking conditions” of the patient X, to the trained model constructed by the medical information processing apparatus 200. The image generating function 16b is an example of a generating unit. In other words, the image generating function 16b is an example of a second obtaining unit configured to input the obtained first MR image to the trained model capable of outputting an MR image corresponding to the time when the radiation irradiation is at a halt in response to an input of an MR image and to obtain the second MR image output by the trained model.

For example, the image generating function 16b is configured to read the trained model stored in the storage circuitry 12. After that, the image generating function 16b is configured to cause the trained model to output the high-quality MR image of the patient X, by inputting the MR image corresponding to the Linac beam-ON time, the irradiation conditions, and the image taking conditions output from the imaging controlling function 16a to the read trained model. Further, the image generating function 16b is configured to send the high-quality MR image of the patient X output from the trained model to the output controlling function 16c.

As explained above, by performing the image generating process using the trained model, the image generating function 16b is configured to generate, from the MR image corresponding to the Linac beam-ON time, the high-quality MR image similar to an MR image corresponding to the Linac beam-OFF time. It should be noted that the image generating process can be used as an image correcting process to correct an MR image having an image distortion and/or image noise to obtain an MR image having no image distortion and/or no image noise.

The output controlling function 16c is configured to cause the high-quality MR image to be output. For example, the output controlling function 16c is configured to output the high-quality MR image of the patient X output from the trained model to one or both of: an IGRT application for implementing IGRT and a synchronized irradiation application for implementing the synchronized irradiation. The IGRT application is configured to implement IGRT by using the high-quality MR image of the patient X. The synchronized radiation application is configured to implement the synchronized irradiation by using the high-quality MR image of the patient X. The output controlling function 16c is an example of an output controlling unit.

Possible output destinations of the high-quality MR image output by the output controlling function 16c are not limited to the IGRT application and the synchronized irradiation application. For example, the output controlling function 16c may cause the display 11 to display the high-quality MR image. Further, the output controlling function 16c may transmit the high-quality MR image to an external apparatus connected to the radiation therapy apparatus 100 via the network NW10. Further, the output controlling function 16c may store the high-quality MR image into the storage circuitry 12 or a portable recording medium.

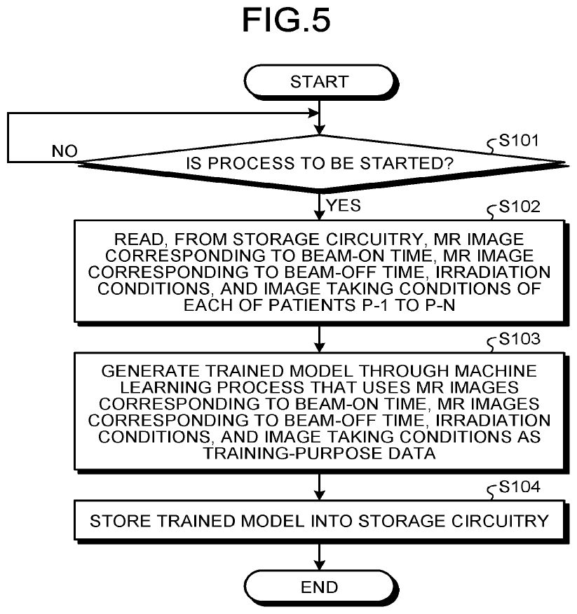

Next, a processing procedure performed by the medical information processing apparatus 200 according to the embodiment will be explained, with reference to FIG. 5. FIG. 5 is a flowchart illustrating a processing procedure performed by the medical information processing apparatus 200 according to the embodiment. The processes illustrated in FIG. 5 are started, for example, when an instruction to start the machine learning process is received from the operator.

As illustrated in FIG. 5, for example, in the medical information processing apparatus 200, the processing circuitry 25 determines that the process is to be started upon receipt of an instruction to start the machine learning process from the operator (step S101: Yes). Unless such an instruction is received, the processing circuitry 25 is in a standby state (step S101: No).

Subsequently, the processing circuitry 25 reads, from the storage circuitry 24, an MR image corresponding to the beam-ON time, an MR image corresponding to the beam-OFF time, the irradiation conditions, and the image taking conditions of each of the patients P-1 to P-N (step S102). After that, the processing circuitry 25 generates a trained model by performing the machine learning process that uses, as training-purpose data, the MR images corresponding to the beam-ON time, the MR images corresponding to the beam-OFF time, the irradiation conditions, and the image taking conditions (step S103). After that, the processing circuitry 25 stores the generated trained model into the storage circuitry 24 (step S104) and ends the process. The trained model stored in the storage circuitry 24 is transferred to the storage circuitry 12 of the radiation therapy apparatus 100 with arbitrary timing.

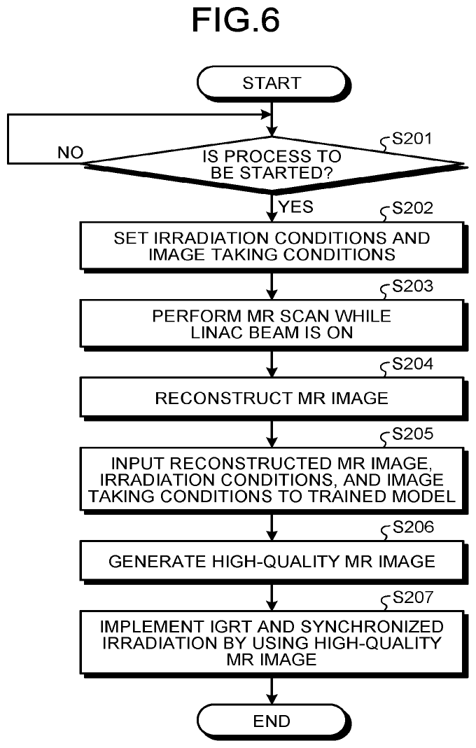

Next, a processing procedure performed by the radiation therapy apparatus 100 according to the embodiment will be explained, with reference to FIG. 6. FIG. 6 is a flowchart illustrating the processing procedure performed by the radiation therapy apparatus 100 according to the embodiment. The processes illustrated in FIG. 6 are started, for example, when an instruction to start the radiation therapy is received from the operator.

As illustrated in FIG. 6, for example, in the radiation therapy apparatus 100, the processing circuitry 16 determines that the process is to be started, upon receipt of an instruction to start the radiation therapy from the operator (step S201: Yes). Unless such an instruction is received, the processing circuitry 16 is in a standby state (step S201: No).

Subsequently, the processing circuitry 16 sets irradiation conditions and image taking conditions for the patient X (step S202). After that, the processing circuitry 14 performs an MR scan while the Linac beam is ON (step S203). Further, the processing circuitry 15 reconstructs an MR image (step S204).

After that, by inputting the reconstructed MR image, the set irradiation conditions, and the set image taking conditions to the trained model (step S205), the processing circuitry 16 generates a high-quality MR image of the patient X (step S206). The processing circuitry 13 implements IGRT or the synchronized irradiation by using the high-quality MR image of the patient X (step S207) and ends the processes in FIG. 6 when the radiation therapy is completed.

Although FIG. 6 illustrates the example in which the MR scan is performed while the Linac beam is ON, possible embodiments are not limited to this example. For instance, the radiation therapy apparatus 100 may, in some situations, perform an MR scan while switching the Linac beam on and off. In those situations, in the radiation therapy apparatus 100, the image generating function 16b judges whether an MR image obtained by the imaging controlling function 16a is an image corresponding to the Linac beam-ON time or an image corresponding to the Linac beam-OFF time. Further, when the image is an image corresponding to the Linac beam-ON time, the image generating function 16b performs the processes at steps S204 through S207. On the contrary, when the image is an image corresponding to the Linac beam-OFF time, the image generating function 16b does not perform the processes at steps S204 through S207. In other words, when the image is an image corresponding to the Linac beam-OFF time, the output controlling function 16c outputs the MR image obtained by the imaging controlling function 16a without performing any process thereon.

As explained above, in the radiation therapy apparatus 100 according to the embodiment, the imaging controlling function 16a is configured to obtain the first Magnetic Resonance (MR) image corresponding to the Linac beam-ON time of the first patient. The image generating function 16b is configured to generate the second MR image corresponding to the Linac beam-OFF time, by inputting the first MR image to the trained model that has been trained with respect to the plurality of second patients by using the MR images corresponding to the Linac beam-ON time and the MR images corresponding to the Linac beam-OFF time. The output controlling function 16c is configured to output the second MR image. The radiation therapy apparatus 100 is able to provide the high-quality MR image in the radiation therapy. For example, the radiation therapy apparatus 100 is able to provide the high-quality MR image in which the image distortions and the image noise caused by the Linac beam have been reduced, by converting the MR image corresponding to the Linac beam-ON time into the MR image similar to an MR image corresponding to the Linac beam-OFF time.

Further, by providing the high-quality MR image, the radiation therapy apparatus 100 is able to enhance treatment precision levels of the radiation therapy. For example, IGRT and the synchronized irradiation have been used as techniques for enhancing treatment precision levels of radiation therapy. However, because MR images corresponding to the Linac beam-ON time contain image distortions and/or image noise, precision levels in estimating the positions of a target site such as a tumor and organs serving as landmarks might be degraded. As a result, there would be a possibility that the precision levels of IGRT and the synchronized irradiation might be degraded. In contrast, the radiation therapy apparatus 100 according to the present embodiment is configured to generate the high-quality MR image in which the image distortions and the image noise caused by the Linac beam have been reduced and configured to implement IGRT or the synchronized irradiation by using the generated high-quality MR image. With this arrangement, the radiation therapy apparatus 100 is able to prevent the degradation of the precision levels of IGRT and the synchronized irradiation that may be caused while the Linac beam is ON.

Further, for example, in the radiation therapy apparatus 100, the imaging controlling function 16a is configured to obtain the irradiation conditions of the Linac beam used at the time of taking the MR image corresponding to the Linac beam-ON time of the patient X. The trained model is further trained by using the irradiation conditions of the Linac beam at the time of taking the MR images corresponding to the Linac beam-ON time of the patients P-1 to P-N. The image generating function 16b is further configured to generate the high-quality MR image by inputting, to the trained model, the irradiation conditions of the Linac beam at the time of taking the MR image corresponding to the Linac beam-ON time of the patient X. In other words, the image generating function 16b is configured to input the irradiation conditions together with the first MR image and to obtain the second MR image output by the trained model. With these arrangements, the radiation therapy apparatus 100 is expected to provide an MR image having higher image quality, by using the irradiation conditions of the Linac beam.

Further, for example, in the radiation therapy apparatus 100, the imaging controlling function 16a is configured to obtain the image taking conditions of the MR image corresponding to the Linac beam-ON time of the patient X. The trained model is further trained by using the image taking conditions of the MR images corresponding to the Linac beam-ON time of the patients P-1 to P-N. The image generating function 16b is configured to generate the high-quality MR image by inputting, to the trained model, the image taking conditions of the MR image corresponding to the Linac beam-ON time of the patient X. In other words, the image generating function 16b is configured to input the image taking condition together with the first MR image and to obtain the second MR image output by the trained model. With these arrangements, the radiation therapy apparatus 100 is expected to provide an MR image having a higher image quality by using the image taking conditions of the MR image.

In the above embodiment, the example was explained using the trained model configured to output the MR image corresponding to the time when the radiation irradiation is at a halt in response to the input of the MR image corresponding to the time when the radiation is being irradiated; however, possible embodiments are not limited to this example. It is sufficient when the processing circuitry 16 of the radiation therapy apparatus 100 is configured to perform, on the first MR image, an image processing process to reduce impacts of the radiation, so as to perform a process of obtaining the second MR image in which the impacts of the radiation have been reduced. For example, the processing circuitry 16 may use a trained model configured to output an MR image from which image distortions and image noise have been removed in response to an input of an MR image. Alternatively, the processing circuitry 16 may be configured, without using any trained model, to generate a second MR image from which the impacts of the radiation have been reduced, by performing a prescribed calculating process on the first MR image.

### First Modification Example

In the embodiments above, the example was explained in which the irradiation conditions and the image taking conditions are used as the training-purpose data; however, possible embodiments are not limited to this example. It is also possible to use information other than the irradiation conditions and the image taking conditions as the training-purpose data.

For example, it is possible to use, as the training-purpose data, at least one selected from among: pieces of information indicating the irradiated site, a fixture, and a physical characteristic of the patient. In this situation, the irradiated site includes at least one of: a target organ (lung, liver, kidney, pancreas, etc.) onto which the Linac beam is irradiated; the position of the organ; and the shape of the organ. The fixture corresponds, for example, to an accessory in use (e.g., a head shell or an arm rest) that was used at the time of taking the MR image. The physical characteristic denotes the physique or the age of the patient.

In other words, the imaging controlling function 16a is further configured to obtain at least one selected from among: the information indicating the irradiation site of the Linac beam (the radiation) at the time of taking the first MR image; the information indicating the fixture used at the time of taking the first MR image; and the information indicating the physical characteristic of the first patient (the imaged person in the first MR image). The trained model is further trained by using at least one selected from among: information indicating the irradiation sites of the Linac beam at the time of taking the MR images; information indicating fixtures used at the time of taking the MR images; and information indicating physical characteristics of the second patients. The image generating function 16b is further configured to generate a high-quality MR image by inputting the obtained one or more pieces of information to the trained model. With these arrangements, the radiation therapy apparatus 100 is expected to provide an MR image having higher quality, by further using the information other than the irradiation conditions and the image taking conditions.

### Second Modification Example

Further, in the above embodiments, the example was explained in which the MR images taken by the MRI function of the radiation therapy apparatus 100 are used as the training data; however, possible embodiments are not limited to this example. For instance, it is also possible to use, as the training data, MR images taken by an MRI apparatus (which may be referred to as an MRI dedicated apparatus) that is not provided with the Linac (radiation irradiation) function.

To reduce impacts imposed by the static magnetic field on the Linac functions, MR-Linac apparatuses have installed therein a static magnetic field magnet having a lower magnetic field intensity (e.g., approximately 1.5 tesla) than that of MRI dedicated apparatuses. For this reason, a trained model according to a second modification example is trained by using MR images taken of the second patients by a magnetic resonance imaging apparatus that is not provided with the Linac functions, as MR images corresponding to the Linac beam-OFF time (training data). With these arrangements, the radiation therapy apparatus 100 is able to construct the trained model capable of outputting an MR image having higher image quality than MR images corresponding to the Linac beam-OFF time.

### Third Modification Example

Further, the radiation therapy apparatus 100 is able to irradiate radiation by using the high-quality MR image obtained in the above embodiments.

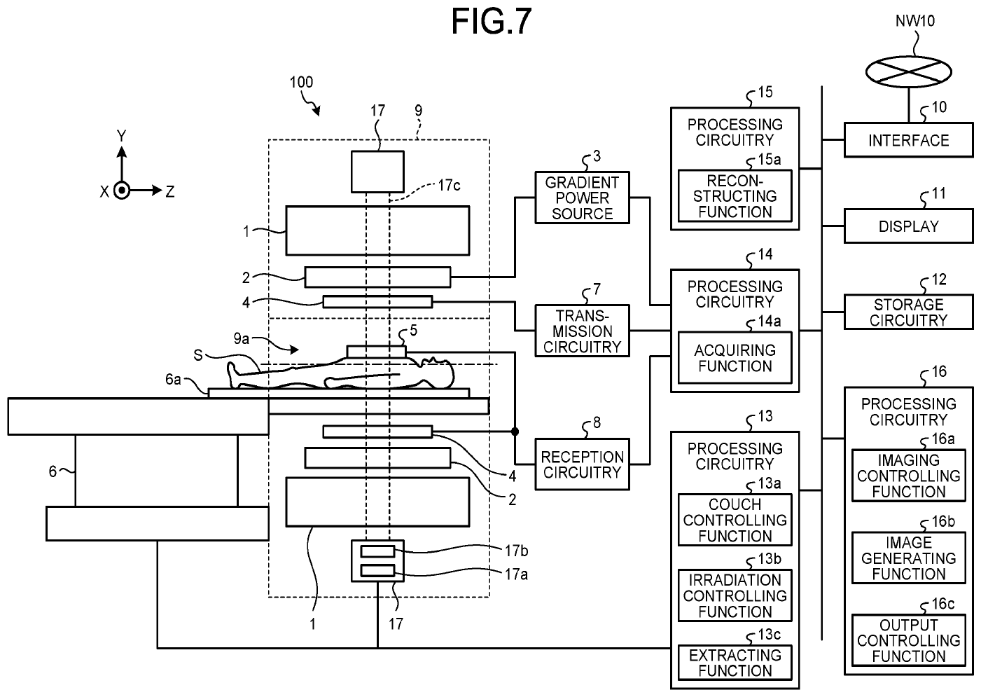

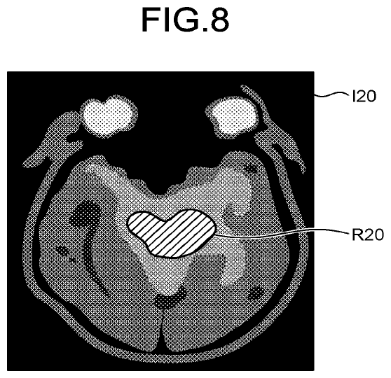

A configuration and processes of the radiation therapy apparatus 100 according to a third modification example of the embodiment will be explained, with reference to FIGS. 7 and 8. FIG. 7 is a diagram illustrating an exemplary configuration of the radiation therapy apparatus 100 according to the third modification example of the embodiment. FIG. 8 is a drawing for explaining the processes performed by the radiation therapy apparatus 100 according to the third modification example of the embodiment.

As illustrated in FIG. 7, the radiation therapy apparatus 100 according to the third modification example of the embodiment has a similar configuration to that of the radiation therapy apparatus 100 illustrated in FIG. 1 and is different in that the processing circuitry 13 further executes an extracting function 13c and for a part of the processes performed by the irradiation controlling function 13b. Thus, the third modification example of the embodiment will be explained while a focus is placed on the differences from the above embodiment. Some of the constituent elements having the same functions as those described in the above embodiment will be referred to by using the same reference characters as those in FIG. 1, and the explanations thereof will be omitted.

More specifically, the extracting function 13c is configured to extract a treatment site of the patient from the second MR image obtained by the image generating function 16b (the second obtaining unit). Further, the irradiation controlling function 13b is configured to irradiate radiation on the treatment site extracted by the extracting function 13c. The extracting function 13c is an example of an extracting unit. Further, the irradiation controlling function 13b is an example of an irradiation controlling unit.

As illustrated in FIG. 8, for example, the extracting function 13c is configured to read the MR image I20 generated by the image generating function 16b. Further, the extracting function 13c is configured to extract a region R20 corresponding to the treatment site by performing a segmentation process on the MR image I20. As for the technique for extracting the region corresponding to the treatment site, possible techniques are not limited to segmentation techniques. It is possible to apply thereto any of publicly-known region extraction techniques, as appropriate.

Further, the irradiation controlling function 13b is configured to irradiate radiation based on the irradiation conditions, on the region R20 extracted by the extracting function 13c. Except for the radiation irradiation on the region R20, the processes performed by the irradiation controlling function 13b are the same as the processes performed by the irradiation controlling function 13b explained with reference to FIG. 1.

As a result, the radiation therapy apparatus 100 according to the third modification example of the embodiment is able to irradiate the radiation by using the high-quality MR image.

In the above third modification example also, the radiation therapy apparatus 100 may, similarly to the above embodiment, perform an MR scan while switching the Linac beam on and off in some situations. In those situations, similarly to the above embodiment, the image generating function 16b is able to judge whether the image generating process using the trained model is necessary or not, depending on whether the MR image obtained by the imaging controlling function 16a is an image corresponding to the Linac beam-ON time or an image corresponding to the Linac beam-OFF time. FIG. 8 illustrates a case of extracting a region corresponding to the treatment site (e.g., a tumor) but is not limited thereto, and a region corresponding to peritumoral tissue (e.g., organ-at-risk) may also be extracted.

### Other Embodiments

It is possible to carry out the present disclosure in other various modes besides the embodiments described above. A medical image processing apparatus

Further, for example, the processing functions according to the above embodiment may be included in a medical image processing apparatus. Further, by providing the medical image processing apparatus in the network NW10, it is also possible to provide the image generating process described above as a cloud service.

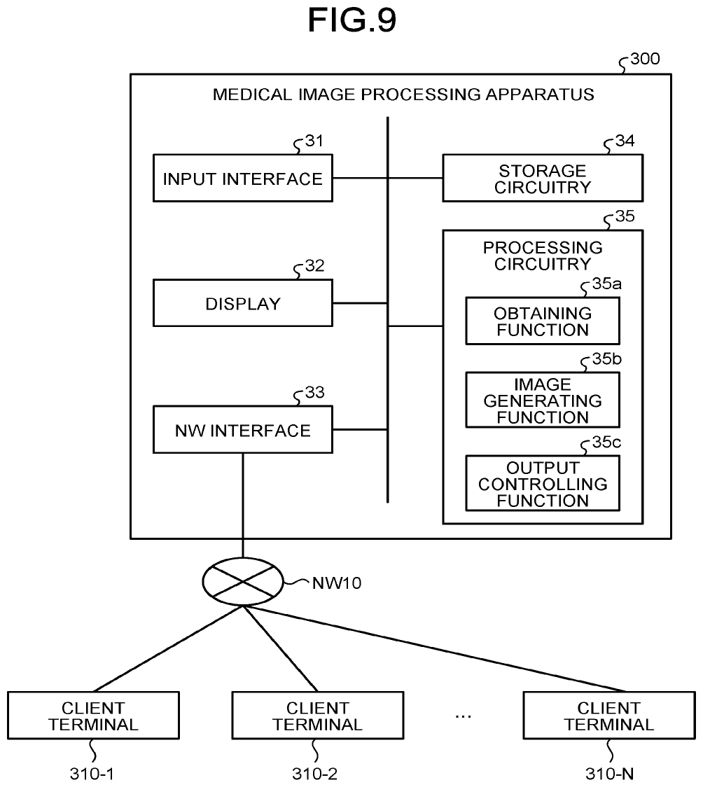

FIG. 9 is a diagram illustrating an exemplary configuration of the medical image processing apparatus according to the one other embodiment. As illustrated in FIG. 9, for example, at a service center providing the cloud service, a medical image processing apparatus 300 is installed. The medical image processing apparatus 300 is connected to a plurality of client terminals 310-1, 310-2, . . . , and 310-N, via the network NW10. When being referred to without being distinguished from one another, the plurality of client terminals 310-1, 310-2, . . . , and 310-N may collectively be referred to as “client terminals 310”.

The client terminals 310 are information processing terminals operated by a user who uses the cloud service. In this situation, the user is, for example, a medical provider such as a medical doctor or a medical technologist working in a medical facility. For example, the client terminals 310 each correspond to an information processing apparatus such as a personal computer or a workstation or to a radiation therapy apparatus such as MR-Linac. The client terminals 310 each have a client function capable of using the cloud service provided by the medical image processing apparatus 300. The client function is recorded, in advance, in each of the client terminals 310 in the form of a computer-executable program.

The medical image processing apparatus 300 includes an input interface 31, a display 32, a NW interface 33, storage circuitry 34, and processing circuitry 35.

The input interface 31 is configured to receive operations to input various types of instructions and information from the operator. Because the basic configuration of the input interface 31 is the same as the configuration of the input interface 21, the explanations thereof will be omitted.

The display 32 is configured to display various types of information and images. Because the basic configuration of the display 32 is the same as the configuration of the display 22, the explanations thereof will be omitted.

The NW interface 33 is connected to the processing circuitry 35 and is configured to control communication performed between the medical image processing apparatus 300 and the client terminals 310. Because the basic configuration of the NW interface 33 is the same as the configuration of the NW interface 23, the explanations thereof will be omitted.

The storage circuitry 34 is connected to the processing circuitry 35 and is configured to store various types of data therein. Because the basic configuration of the storage circuitry 34 is the same as the configuration of the storage circuitry 24, the explanations thereof will be omitted.

The processing circuitry 35 is configured to control operations of the medical image processing apparatus 300, in accordance with the input operations received from the operator via the input interface 31. For example, the processing circuitry 35 is realized by using a processor.

The processing circuitry 35 is configured to execute an obtaining function 35a, an image generating function 35b, and an output controlling function 35c. The processing functions executed by the processing circuitry 35 are, for example, recorded in the storage circuitry 34 in the form of computer-executable programs. The processing circuitry 35 is configured to read and execute the programs, so as to realize the functions corresponding to the read programs.

In this situation, for example, by operating one of the client terminals 310, the user inputs an instruction indicating that the MR image corresponding to the Linac beam-ON time taken by the MR-Linac be transmitted to (uploaded into) the medical image processing apparatus 300. When the instruction is input, the client terminal 310 transmits the MR image corresponding to the Linac beam-ON time with respect to the patient X undergoing the radiation therapy, to the medical image processing apparatus 300.

After that, in the medical image processing apparatus 300, the obtaining function 35a is configured to obtain the MR image corresponding to the Linac beam-ON time, by receiving the MR image corresponding to the Linac beam-ON time transmitted from the client terminal 310.

Subsequently, the image generating function 35b is configured to generate a high-quality MR image similar to an MR image corresponding to the Linac beam-OFF time, by inputting the MR image corresponding to the Linac beam-ON time obtained by the obtaining function 35a to the trained model. Because the trained model is the same as the trained model described in the above embodiment, the explanations thereof will be omitted.

Further, the output controlling function 35c is configured to cause the high-quality MR image generated by the image generating function 35b to be transmitted to (or to be downloaded into) the client terminal 310 from which the MR image corresponding to the Linac beam-ON time was transmitted.

As explained above, the medical image processing apparatus 300 according to the one other embodiment is able to provide the high-quality MR image in the radiation therapy. Possible transmission destinations of the high-quality MR image are not limited to the client terminal 310 from which the MR image corresponding to the Linac beam-ON time was transmitted. It is possible to transmit the high-quality MR image to arbitrary apparatuses.

Further, the medical image processing apparatus 300 does not necessarily need to be provided as a cloud service. For example, the medical image processing apparatus 300 may be provided as a medical doctor's terminal in the facility. In that situation, the medical image processing apparatus 300 does not necessarily have to be connected to the network NW10.

Further, the medical image processing apparatus 300 may further include the learning function 25a illustrated in FIG. 2. In that situation, the medical image processing apparatus 300 is able to update the trained model, by performing a machine learning process again while using the MR image corresponding to the Linac beam-ON time transmitted from the client terminal 310 as new training-purpose data.

The constituent elements of the apparatuses and devices in the drawings are based on functional concepts. Thus, it is not necessarily required to physically configure the constituent elements as indicated in the drawings. In other words, specific modes of distribution and integration of the apparatuses and devices are not limited to those illustrated in the drawings. It is acceptable to functionally or physically distribute or integrate all or a part of the apparatuses and devices in any arbitrary units, depending on various loads and the status of use. Further, all or an arbitrary part of the processing functions performed by the apparatuses and devices may be realized by a CPU and a program analyzed and executed by the CPU or may be realized as hardware using wired logic.

Further, with regard to the processes explained in the embodiments and the modification examples described above, it is acceptable to manually perform all or a part of the processes described as being performed automatically. Conversely, by using a publicly-known method, it is also acceptable to automatically perform all or a part of the processes described as being performed manually. Further, unless noted otherwise, it is acceptable to arbitrarily modify any of the processing procedures, the controlling procedures, specific names, and various information including various types of data and parameters that are presented in the above text and the drawings.

In addition, it is possible to realize the medical image processing methods explained in the embodiments and the modification examples described above, by causing a computer such as a personal computer or a workstation to execute a medical image processing program prepared in advance. The medical image processing program may be distributed via a network such as the Internet. Further, the medical image processing program may be executed, as being recorded on a computer-readable recording medium such as a hard disk, a flexible disk (FD), a Compact Disk Read-Only Memory (CD-ROM), a Magneto Optical (MO) disk, a Digital Versatile Disk (DVD) or the like and being read by a computer from the recording medium.

According to at least one aspect of the embodiments described above, it is possible to provide the high-quality MR images in the radiation therapy.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A radiation therapy apparatus comprising processing circuitry configured:
to obtain a first Magnetic Resonance (MR) image corresponding to time when radiation is being irradiated; and
to perform, on the first MR image, an image processing process to reduce an impact of the radiation, so as to obtain a second MR image in which the impact of the radiation is reduced.

2. The radiation therapy apparatus according to claim 1, wherein
the processing circuitry inputs the first MR image to a trained model configured to output an MR image corresponding to time when radiation irradiation is at a halt in response to an input of an MR image corresponding to time when radiation is being irradiated, and
the processing circuitry obtains the MR image output by the trained model as the second MR image.

3. The radiation therapy apparatus according to claim 1, wherein the processing circuitry further extracts a treatment site of a patient from the obtained second MR image.

4. The radiation therapy apparatus according to claim 3, wherein the processing circuitry further irradiates radiation onto the extracted treatment site.

5. The radiation therapy apparatus according to claim 1, wherein
the processing circuitry obtains an irradiation condition of the radiation,
the processing circuitry inputs the irradiation condition together with the first MR image to the trained model, and
the processing circuitry obtains the second MR image output by the trained model.

6. The radiation therapy apparatus according to claim 1, wherein
the processing circuitry obtains an image taking condition of the first MR image,
the processing circuitry inputs the image taking condition together with the first MR image to the trained model, and
the processing circuitry obtains the second MR image output by the trained model.

7. The radiation therapy apparatus according to claim 1, wherein
the processing circuitry is further configured:
to obtain at least one of: information indicating a radiation irradiated site at a time of taking the first MR image; information indicating a fixture used at the time of taking the first MR image; and information indicating a physical characteristic of an imaged person in the first MR image; and
to obtain the second MR image by inputting the obtained one or more pieces of information to the trained model.

8. The radiation therapy apparatus according to claim 1, wherein the trained model is trained by using an MR image taken by a magnetic resonance imaging apparatus that is not provided with a function to irradiate radiation.

9. The radiation therapy apparatus according to claim 1, wherein the processing circuitry further outputs the second MR image to one or both of: an application for implementing image-guided radiotherapy; and an application for implementing synchronized irradiation.

10. A medical image processing apparatus comprising processing circuitry configured:
to obtain a first Magnetic Resonance (MR) image corresponding to time when radiation is being irradiated; and
to perform, on the first MR image, an image processing process to reduce an impact of the radiation, so as to obtain a second MR image in which the impact of the radiation is reduced.

11. A medical image processing method comprising:
obtaining a first Magnetic Resonance (MR) image corresponding to time when radiation is being irradiated; and
performing, on the first MR image, an image processing process to reduce an impact of the radiation, so as to obtain a second MR image in which the impact of the radiation is reduced.
